SEQUENCE LISTING (8 sequences)

SEQ ID NO: 1 - protein, 99 aa, Artificial Sequence
  Synthesized
TTCCTCACATCTTACGACGGCAGTCTCTCCAGAGTCCTCAGCATGACCTGTTAGTAACTGAAGATAAGGGTTGCGCTCGTTATGGCACTTAAGCCGACA

SEQ ID NO: 2 - protein, 100 aa, Artificial Sequence
  Synthesized
GGCACTTAAGCCGACACCTCACGGCACGAGCTGACGACAACCATGCAGCACCTTCACAGCGGTGATTGCTCACTGACATGTTTCCACATCATTCCACTGC

SEQ ID NO: 3 - protein, 100 aa, Artificial Sequence
  Synthesized
CACTTTCGAGCATCAGTGTCAGTTGCAGTCCAGTGAGCTGCCTTCGCAATCGGAGTTCTTCGTGATATCTAAGCATTTCACCGCTACACCACGAATTCCG

SEQ ID NO: 4 - protein, 100 aa, Artificial Sequence
  Synthesized
TATCTAATCCTGTTTGATACCCACACTTTCGAGCATCAGTGTCAGTTGCAGTCCAGTGAGCTGCCTTCGCAATCGGAGTTCTTCGTGATATCTAAGCATT

SEQ ID NO: 5 - protein, 100 aa, Artificial Sequence
  Synthesized
GCTCCCTTTAAACCCAATAAATCCGGATAACGCTCGGATCCTCCGTATTACCGCGGCTGCTGGCACGGAGTTAGCCGATCCTTATTCATATTATACATAC

SEQ ID NO: 6 - protein, 100 aa, Artificial Sequence
  Synthesized
TTGACGGGCGGTGTGTACAAGGCCCGGGAACGTATTCACCGCGCCGTGGCTGATGCGCGATTACTAGCGAATCCAGCTTCACGAAGTCGGGTTGCAGACT

SEQ ID NO: 7 - protein, 115 aa, Artificial Sequence
  Synthesized
CCTCAGGTCATCCGGAAGCTTTTCAACGCTTATCGGTTCGGTCCTCCAGTTAGTGTTACCTAACCTTCAACCTGCCCAAGGGTAGATCACTTGGTTTCGCGTCTACTCCTTCCGA

SEQ ID NO: 8 - protein, 110 aa, Artificial Sequence
  Synthesized
TCACAGTACTGGTTCGCTATCGGTCTCTCGGGAGTATTTAGCCTTACCGGATGGTCCCGGCTGGTTCACGCAGAATTCCTCGTGCTCCGCGCTACTCAGGATACCACTAC

(19) United States
(12) Patent Application Publication
(10) Pub. No.: US 2022/0235404 A1
(43) Pub. Date: Jul. 28, 2022

(54) METHODS FOR DETECTION OF RARE DNA SEQUENCES IN FECAL SAMPLES

(71) Applicant: AMERICAN MOLECULAR LABORATORIES, INC., Vernon Hills, IL (US)

(72) Inventors: Hongjun ZHANG, Vernon Hills, IL (US); Yi ZHOU, Vernon Hills, IL (US); Rajarao KAKUTURU, Vernon Hills, IL (US); Zhaozhong CHONG, Vernon Hills, IL (US)

(21) Appl. No.: 17/613,622

(22) PCT Filed: May 26, 2020
(86) PCT No.: PCT/US20/34511

§ 371 Date: Nov. 23, 2021

Related U.S. Application Data
(60) Provisional application No. 62/852,018, filed on May 23, 2019.

(51) Int. Cl. C12Q 1/6827; C12Q 1/6886; C12Q 1/689; C12Q 1/6806

(52) U.S. Cl. CPC C12Q 1/6827; C12Q 1/6806; C12Q 1/689; C12Q 1/6886

(57) ABSTRACT

The disclosure provides methods and materials for identifying a low copy number DNA sequence in a fecal sample, such as a low copy number DNA sequence from a pathogenic bacterial species or genetic variant associated with disease.

### CROSS-REFERENCE TO RELATED APPLICATIONS

This application claims the benefit of and priority to U.S. Patent Application No. 62/852,018, filed May 23, 2019, which is incorporated herein by reference in its entirety.

### FIELD

The present disclosure generally provides methods and compositions for detecting rare DNA sequences in fecal samples.

### SEQUENCE LISTING

The instant application contains a Sequence Listing which has been submitted electronically in ASCII format and is hereby incorporated by reference in its entirety. Said ASCII copy, created on May 26, 2020, is named “116110-5005-WO_ST25_Sequence_Listing” and is 8 kilobytes in size.

### BACKGROUND

Feces is a readily accessible and abundant source of metabolic waste. It contains trillions of microorganisms that reside in the mammalian gastrointestinal tract, along with millions of host cells, including macrophages and lymphocytes that migrate between the gut lumen and blood circulation. Mounting knowledge on human gut microbiota indicate that the composition of bacterial taxa in gastrointestinal tract is important for homeostasis and disease, with numerous disorders from the neurologic, psychiatric, respiratory, cardiovascular, gastrointestinal, hepatic, autoimmune, metabolic and oncologic spectra. Moreover, aberrant host cells present in feces have genetic signatures of disease. Feces thus afford a valuable, noninvasive source of biological material for pathogen detection, gut microbiota analysis, and disease diagnosis, and hold invaluable potential for applications in diagnosis, disease prediction and therapeutic intervention.

Conventional methods of fecal analysis comprise microbial culture combined with biochemical, immunochemical, genetic (DNA or RNA), and/or microscopic analysis. However, culture-based methods of fecal material are limited because some fecal microbes are difficult to culture, and the large fraction of normal, symbiotic bacteria in feces presents a high background that can preclude detection of rarer species. Moreover, microbial culture does not facilitate analysis of small amounts of host material in fecal samples.

Certain genetic techniques amplify the target material and therefore do not require culturing, including PCR, quantitative PCR, and DNA/RNA sequencing. Of particular note, next-generation sequencing (NGS) facilitates the assessment of genes and genomes in a specimen with complex microbial communities at the sequence level. However, these methods are also complicated by the high background of DNA from normal bacteria present in the sample. The largest portion of DNA in feces is from normal gut microbiota, representing 60-70% of the dry weight of feces. Thus, when shotgun NGS is performed on a fecal sample, a huge amount of background, non-informative reads are generated from normal gut microbiota, complicating or precluding accurate detection and analysis, and increasing cost. Moreover, feces is one of the most difficult biological specimens to obtain high-quality DNA and RNA for molecular analysis, since it contains large quantities of nucleases that degrade the DNA and RNA, and various nucleic acid contaminants that interfere with subsequent molecular analysis, and many inhibitors hampering subsequent PCR amplification and NGS procedures.

Accordingly, there is a need in the art for methods of detecting rare (e.g. low copy number) DNA species in fecal samples, and compositions for performing those methods.

### SUMMARY

The present disclosure relates to methods and materials for identifying a low copy number DNA sequence in a fecal sample, including, for example, a low copy number DNA sequence from a pathogenic bacterial species. In some embodiments, the disclosure relates to identifying a low copy number genetic variant associated with disease, such as cancer, in a fecal sample. In further embodiments, the disclosure relates to methods of enriching a low copy number DNA sequence for detection by quantitative or semi-quantitative means, or for detection by sequencing. The disclosure also relates to methods and compositions for preparing a sequencing library comprising low copy number DNA sequences from a fecal sample. Additionally, the disclosure relates to compositions and kits for depleting abundant bacterial species in a sample using labeled oligonucleotides.

In some embodiments of each or any of the above or below mentioned embodiments, the disclosure provides methods and compositions for identifying a low copy number DNA sequence in a fecal sample comprising obtaining the fecal sample from a subject, extracting DNA from the fecal sample to obtain a DNA preparation, hybridizing a labeled oligonucleotide probe to non-pathogenic bacterial DNA sequences in the DNA preparation to form a hybridization complex, depleting the hybridization complex from the DNA preparation, and identifying the presence of the low copy number DNA sequence in the DNA preparation, wherein identification of the low copy number DNA sequence in the DNA preparation indicates that the low copy number DNA sequence is present in the fecal sample.

In some embodiments of each or any of the above or below mentioned embodiments, the disclosure provides methods and compositions for identifying low copy number DNA sequences from a pathogenic bacterial species, such as H. pylori DNA sequences. In other embodiments, the low copy number DNA sequence identified according to the disclosure is a human DNA sequence, such as a disease associated genetic variant. In certain embodiments, the disease associated genetic variant is associated with cancer, e.g., colon cancer.

In some embodiments of each or any of the above or below mentioned embodiments, the disclosure provides methods and materials for depleting DNA sequences of one or more non-pathogenic bacterial species present in a fecal sample, wherein the non-pathogenic bacterial species comprise Bacteroides, Clostridium, Faecalibacterium, or a combination thereof. In some embodiments, the non-pathogenic bacterial species comprise Bacteroides, including Bacteroides fragilis, Bacteroides melaninogenicus, Bacteroides oralis, or a combination thereof.

In some embodiments of each or any of the above or below mentioned embodiments, the disclosure provides methods and compositions for identifying a low copy number DNA sequence in a fecal sample comprising hybridizing a labeled oligonucleotide probe to non-pathogenic bacterial DNA sequences in the DNA preparation to form a hybridization complex. In embodiments, the labeled oligonucleotide probe is complementary to a conserved region of the non-pathogenic bacterial DNA. In further embodiments, the labeled oligonucleotide probe comprises a biotin label.

In some embodiments of each or any of the above or below mentioned embodiments, the disclosure further provides methods and materials for depleting DNA sequences of one or more non-pathogenic bacterial species present in a fecal sample, comprising incubating a biotin-labeled hybridization complex with a streptavidin-coated substrate. In certain embodiments, the streptavidin-coated substrate comprises a bead, a column, or a membrane. In some embodiments, the streptavidin-coated substrate comprises a magnetic bead and the hybridization complexes are depleted from the DNA preparation using a magnetic field. In some embodiments, the hybridization complexes of the disclosure are depleted from the DNA preparation using centrifugal force.

In some embodiments of each or any of the above or below mentioned embodiments, the labeled oligonucleotide probe of the disclosure is selected from SEQ ID NO: 1, SEQ ID NO: 2, SEQ ID NO: 3, SEQ ID NO: 4, SEQ ID NO: 5, SEQ ID NO: 6, SEQ ID NO: 7, or SEQ ID NO: 8.

In some embodiments of each or any of the above or below mentioned embodiments, identifying the presence of the low copy number DNA sequence in the DNA preparation comprises sequencing the DNA sequences. In certain embodiments, identifying the presence of the low copy number DNA sequence in the DNA preparation comprises a quantitative PCR reaction. In further embodiments, the sequencing or quantitative PCR reaction is multiplexed with DNA prepared from multiple fecal samples.

In some embodiments of each or any of the above or below mentioned embodiments, extracting DNA from the fecal sample to obtain a DNA preparation comprises bead homogenizing the fecal sample in a lysis buffer, wherein the lysis buffer comprises ingredients capable of breaking a bacterial cell wall, digesting protein, denaturing protein, dispersing fat, precipitating polysaccharides, or a combination thereof. In some embodiments, total DNA is extracted from the fecal sample. In further embodiments, DNA extracted from the fecal sample weighs between about 0.5 grams to about 1.0 grams.

In some embodiments of each or any of the above or below mentioned embodiments, the disclosure provides methods of enriching low copy number DNA sequences in a fecal sample comprising obtaining the fecal sample from a subject, extracting DNA from the fecal sample to obtain a DNA preparation, hybridizing a labeled oligonucleotide probe to non-pathogenic bacterial DNA sequences in the DNA preparation to form a hybridization complex, and depleting the hybridization complex from the DNA preparation.

In some embodiments of each or any of the above or below mentioned embodiments, the disclosure provides methods and compositions for identifying antibiotic resistant H. pylori in a fecal sample comprising obtaining the fecal sample from a subject, extracting DNA from the fecal sample to obtain a DNA preparation, hybridizing a labeled oligonucleotide probe to non-pathogenic bacterial DNA sequences in the DNA preparation to form a hybridization complex, depleting the hybridization complex from the DNA preparation, amplifying a region of H. pylori DNA in the DNA preparation to generate multiple copies of the region of the H. pylori DNA, sequencing the multiple copies of the amplified region of the H. pylori DNA, comparing sequences of the multiple copies of the amplified region of the H. pylori DNA to a reference sequence, identifying the presence of a mutation in the multiple copies of the region of the H. pylori DNA, and determining a number of the multiple copies of the region of the H. pylori DNA with the mutation, wherein antibiotic resistant H. pylori is present in the sample when the number of the multiple copies of the region of the H. pylori DNA with the mutation is above a predetermined amount.

In some embodiments of each or any of the above or below mentioned embodiments, the disclosure provides methods of preparing a next-generation sequencing library comprising low copy number DNA sequences in a fecal sample comprising obtaining the fecal sample from a subject, extracting DNA from the fecal sample to obtain a DNA preparation, hybridizing a labeled oligonucleotide probe to non-pathogenic bacterial DNA sequences in the DNA preparation to form a hybridization complex, depleting the hybridization complex from the DNA preparation, and amplifying one or more amplicons in the depleted DNA preparation to form a NGS sequencing library.

In some embodiments of each or any of the above or below mentioned embodiments, the disclosure provides methods of detecting cancer in a subject comprising; obtaining a fecal sample from a subject, extracting DNA from the fecal sample to obtain a DNA preparation, hybridizing a labeled oligonucleotide probe to non-pathogenic bacterial DNA sequences in the DNA preparation to form a hybridization complex, depleting the hybridization complex from the DNA preparation, and detecting the presence of one or more rare cancer-associated DNA sequences in the sample.

### BRIEF DESCRIPTION OF THE DRAWINGS

FIG. 1 illustrates a process of depleting Bacteroides DNA from a fecal extract by introducing biotinylated probes to form hybridization complexes comprising Bacteroides DNA, and using streptavidin-coated magnetic beads to remove the hybridization complexes with a magnetic field.

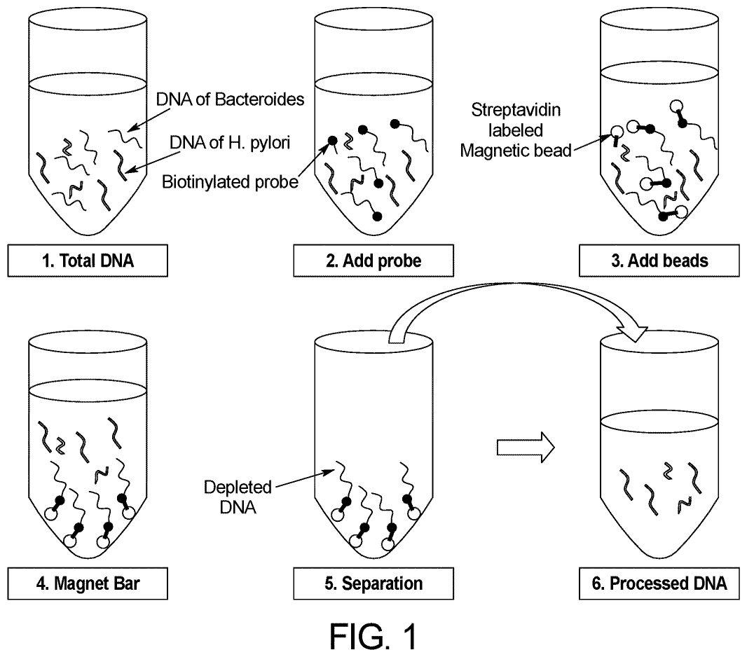

FIG. 2 illustrates a process of depleting Bacteroides DNA by introducing biotinylated probes that form hybridization complexes comprising Bacteroides DNA, and using streptavidin-coated beads to remove the hybridization complexes with a filtration column.

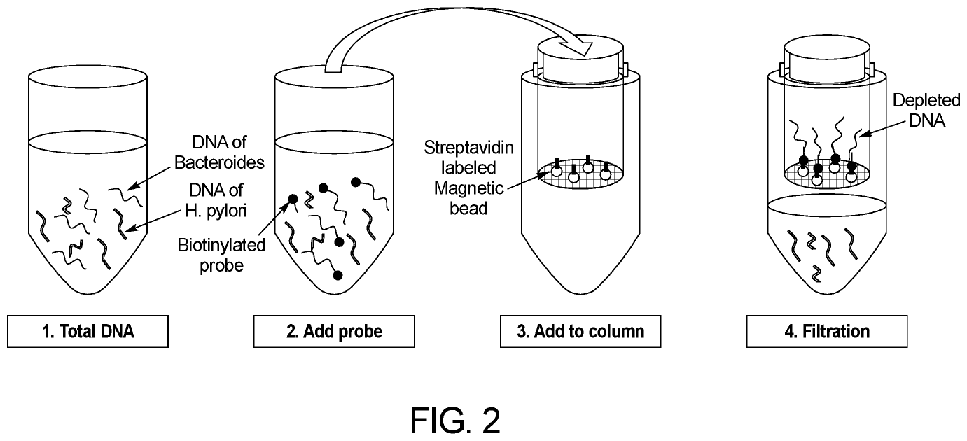

### DETAILED DESCRIPTION

The present disclosure relates to methods and materials for identifying a low copy number DNA sequence in a fecal sample, including, for example, a low copy number DNA sequence from a pathogenic bacterial species. In some embodiments, the disclosure relates to identifying a low copy number genetic variant associated with disease, such as cancer, in a fecal sample.

As disclosed herein, the inventors have surprisingly found that depleting a fecal DNA extract of DNA from non-pathogenic bacteria enables the identification of low copy number DNA sequences in the extract. Thus, the disclosure provides methods and compositions for enriching a low copy number DNA sequence for detection by quantitative or semi-quantitative means, or for detection by sequencing. The disclosure also relates to methods and compositions for preparing a sequencing library comprising low copy number DNA sequences from a fecal sample. Additionally, the disclosure relates to compositions and kits for depleting abundant bacterial species in a sample using labeled oligonucleotides.

Where the term “comprising” is used in the present description and the claims, it does not exclude other elements or steps. For the purposes of the present invention, the term “consisting of” is considered to be a preferred embodiment of the term “comprising”. If hereinafter a group is defined to comprise at least a certain number of embodiments, this is also to be understood to disclose a group, which preferably consists only of these embodiments.

Where numerical values are indicated in the context of the present disclosure the skilled person will understand that the technical effect of the feature in question is ensured within an interval of accuracy, which typically encompasses a deviation of the numerical value given of ±10%, and preferably of ±5%.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

The terms “a,” “an,” “the” and similar referents used in the context of describing the disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope of the disclosure otherwise claimed. No language in the specification should be construed as indicating any non-claimed element essential to the practice of the disclosure.

Groupings of alternative elements or embodiments of the disclosure disclosed herein are not to be construed as limitations. Each group member can be referred to and claimed individually or in any combination with other members of the group or other elements found herein. It is anticipated that one or more members of a group can be included in, or deleted from, a group for reasons of convenience and/or patentability. When any such inclusion or deletion occurs, the specification is deemed to contain the group as modified, thus fulfilling the written description of all Markush groups used in the appended claims.

It is to be understood that the embodiments of the disclosure disclosed herein are illustrative of the principles of the present disclosure. Other modifications that can be employed are within the scope of the disclosure. Thus, by way of example, but not of limitation, alternative configurations of the present disclosure can be utilized in accordance with the teachings herein. Accordingly, the present disclosure is not limited to that precisely as shown and described.

Unless otherwise indicated, the practice of the present invention will employ conventional techniques of molecular biology, biochemistry, microbiology, recombinant DNA, nucleic acid hybridization, genetics, immunology, and oncology which are within the skill of the art. Such techniques are explained fully in the literature. See, for example, Green & Sambrook, MOLECULAR CLONING: A LABORATORY MANUAL, Cold Spring Harbor Laboratory Press (2012); Sambrook, Fritsch & Maniatis, MOLECULAR CLONING: A LABORATORY MANUAL, Second Edition, Cold Spring Harbor Laboratory Press (1989).

Further definitions of terms will be given in the following in the context of which the terms are used. The following terms or definitions are provided solely to aid in the understanding of the invention. These definitions should not be construed to have a scope less than understood by a person of ordinary skill in the art.

As used herein, a “sample” or “fecal sample” or “stool sample” means a sample of feces collected from a subject. A sample may be directly tested or else all or some of the nucleic acid present in the sample may be isolated prior to testing. In yet another example, the sample may be partially purified or otherwise enriched prior to analysis. For example, to the extent that a sample comprises a very diverse cell population, it may be desirable to enrich for a sub-population of particular interest. It is within the scope of the present invention for the target cell population or molecules derived therefrom to be treated prior to testing, for example, inactivation of live virus. It should also be understood that the sample may be freshly harvested or it may have been stored (for example by freezing) prior to testing or otherwise treated prior to testing (such as by undergoing culturing).

As used herein, H. pylori means any of the H. pylori strains known in the art, including for example the strains listed in Table 1.

TABLE 1
H. pylori Strains

| NO | H. pylori Strain Name |
|---|---|
| 1 | Helicobacter pylori 2017 |
| 2 | Helicobacter pylori 2018 |
| 3 | Helicobacter pylori 26695 |
| 4 | Helicobacter pylori 35A |
| 5 | Helicobacter pylori 51 |
| 6 | Helicobacter pylori 52 |
| 7 | Helicobacter pylori 83 |
| 8 | Helicobacter pylori 908 |
| 9 | Helicobacter pylori Aklavik117 |
| 10 | Helicobacter pylori Aklavik86 |
| 11 | Helicobacter pylori B38 |
| 12 | Helicobacter pylori B8 |
| 13 | Helicobacter pylori BM012A |
| 14 | Helicobacter pylori BM012S |
| 15 | Helicobacter pylori Cuz20 |
| 16 | Helicobacter pylori ELS37 |
| 17 | Helicobacter pylori F16 |
| 18 | Helicobacter pylori F30 |
| 19 | Helicobacter pylori F32 |
| 20 | Helicobacter pylori F57 |
| 21 | Helicobacter pylori G27 |
| 22 | Helicobacter pylori Gambia94/24 |
| 23 | Helicobacter pylori HPAG1 |
| 24 | Helicobacter pylori HUP-B14 |
| 25 | Helicobacter pylori India7 |
| 26 | Helicobacter pylori Lithuania75 |
| 27 | Helicobacter pylori OK113 DNA |
| 28 | Helicobacter pylori OK310 |
| 29 | Helicobacter pylori P12 |
| 30 | Helicobacter pylori PeCan18 |
| 31 | Helicobacter pylori PeCan4 |
| 32 | Helicobacter pylori Puno120 |
| 33 | Helicobacter pylori Puno135 |
| 34 | Helicobacter pylori Rif1 |
| 35 | Helicobacter pylori Rif2 |
| 36 | Helicobacter pylori SJM180 |
| 37 | Helicobacter pylori SNT49 |
| 38 | Helicobacter pylori Sat464 |
| 39 | Helicobacter pylori Shi112 |
| 40 | Helicobacter pylori Shi169 |
| 41 | Helicobacter pylori Shi417 |
| 42 | Helicobacter pylori Shi470 |
| 43 | Helicobacter pylori SouthAfrica20 |
| 44 | Helicobacter pylori SouthAfrica7 |
| 45 | Helicobacter pylori UM032 |
| 46 | Helicobacter pylori UM037 |
| 47 | Helicobacter pylori UM066 |
| 48 | Helicobacter pylori UM298 |
| 49 | Helicobacter pylori UM299 |
| 50 | Helicobacter pylori XZ274 |
| 51 | Helicobacter pylori v225d |
| 52 | Helicobacter pylori-strain J99 |
| 53 | Helicobacter pylori HPbs1 |
| 54 | Helicobacter pylori TH2099 |
| 55 | Helicobacter pylori GD63 |
| 56 | Helicobacter pylori HP14039 |
| 57 | Helicobacter pylori HPbs3 |
| 58 | Helicobacter pylori HPbs2 |
| 59 | Helicobacter pylori PMSS1 |
| 60 | Helicobacter pylori ATCC 43504 |
| 61 | Helicobacter pylori H-137 |
| 62 | Helicobacter pylori NCTC12813 |
| 63 | Helicobacter pylori NCTC13345 |
| 64 | Helicobacter pylori NCTC11637 |
| 65 | Helicobacter pylori FDAARGOS 298 |
| 66 | Helicobacter pylori 7.13 R1B |
| 67 | Helicobacter pylori B128 1 |
| 68 | Helicobacter pylori 7.13 D3c |
| 69 | Helicobacter pylori 7.13 D2b |
| 70 | Helicobacter pylori 26695 dR |
| 71 | Helicobacter pylori dRdM2addM2 |
| 72 | Helicobacter pylori dRdM1 |
| 73 | Helicobacter pylori HPJP26 |
| 74 | Helicobacter pylori G272 |
| 75 | Helicobacter pylori PMSS1 |
| 76 | Helicobacter pylori 7.13 D3b |
| 77 | Helicobacter pylori 7.13 D3a |
| 78 | Helicobacter pylori 7.13 D2c |
| 79 | Helicobacter pylori HP42k |
| 80 | Helicobacter pylori FDAARGOS 300 |
| 81 | Helicobacter pylori 7.13 R1c |
| 82 | Helicobacter pylori 7.13 R1a |
| 83 | Helicobacter pylori 7.13 R2c |
| 84 | Helicobacter pylori 7.13 R2a |
| 85 | Helicobacter pylori 7.13 R2b |
| 86 | Helicobacter pylori 7.13 D2a |

Denaturation refers to the process by which a double-stranded nucleic acid is converted into its constituent single strands. Denaturation can be achieved, for example, by the use of high temperature, low ionic strength, acidic or alkaline pH, and/or certain organic solvents. Methods for denaturing nucleic acids are well-known in the art.

Hybridization (sometimes called annealing) refers to the process by which complementary, single-stranded nucleic acids form a double-stranded structure, or duplex, mediated by hydrogen-bonding between complementary bases in the two strands.

Hybridization conditions are those values of, for example, temperature, ionic strength, pH and solvent which will allow annealing to occur. Many different combinations of the above-mentioned variables will be conducive to hybridization. Appropriate conditions for hybridization are well-known in the art, and will generally include an ionic strength of 50 mM or higher monovalent and/or divalent cation at neutral or near-neutral pH.

A hybridization mixture is a composition containing single-stranded nucleic acid at the appropriate temperature, pH and ionic strength to allow annealing to occur between molecules sharing regions of complementary sequence.

A duplex refers to a double-stranded polynucleotide.

As used herein, a “probe sequence” is a nucleic acid capable of binding to a target nucleic acid of complementary sequence through one or more types of chemical bonds, usually through complementary base pairing, usually through hydrogen bond formation, thus forming a duplex structure. The probe binds or hybridizes to a “probe binding site.” A probe may include natural (i.e. A, G, C, or T) or modified bases (7-deazaguanosine, inosine, etc.). A probe can be a single stranded oligonucleotide. Oligonucleotide probes can be synthesized or produced from naturally occurring polynucleotides. In addition, the bases in a probe can be joined by a linkage other than a phosphodiester bond, so long as it does not interfere with hybridization.

An oligonucleotide is a short nucleic acid, generally DNA and generally single-stranded. Generally, an oligonucleotide will be shorter than 200 nucleotides, more particularly, shorter than 100 nucleotides, most particularly, 50 nucleotides or shorter.

As used herein, a “hybridization complex” is a complex between a probe sequence and a DNA sequence extracted from a fecal sample. For example, a hybridization complex is a complex between a probe sequence that is complementary to a Bacteroides DNA sequence, wherein the probe sequence is bound to the target Bacteroides DNA sequence. In embodiments, a hybridization complex comprises a probe sequence hybridized to single stranded DNA. In other embodiments, a hybridization complex hybridization complex comprises a probe sequence hybridized to double stranded DNA, wherein the probe displaces a region of the double stranded DNA to which it is complementary.

As used herein, “quantitative PCR” or “qPCR” or “quantitative real time PCR” refers to methods of monitoring the amplification of a DNA segment in a sample in real time to determine the level of the DNA segment in the sample.

In embodiments, the methods of the disclosure comprise obtaining a fecal sample from a subject and extracting DNA from the sample. Feces of any animal can be tested in various embodiments disclosed herein. Samples may be collected by any readily available means, e.g., at a point of care facility by medical professionals, or a by the subject using an at home collection kit. In embodiments, samples are kept refrigerated until testing. In embodiments, preparation of the fecal sample can be accomplished using any of the known methods in the art. For example the soluble portion of the sample can be collected using filtration, centrifugation, or simple mixing followed by gravimetric settling.

Fecal samples can be collected and prepared in many ways. For example, in some embodiments the fecal sample comprises a stool supernatant prepared from a stool homogenate. In some embodiments, the methods comprise exposing the fecal sample to a condition that denatures proteins and nucleic acids before extracting bacterial DNA. For example, some embodiments provide that the condition that denatures nucleic acids comprises heating at 90° C. for 10 minutes.

In some embodiments, the fecal sample is lysed to extract its DNA content in a buffer formulated with proportional amounts of Tris-HCl buffer, ethylenediaminetetraacetic acid (EDTA), NaCl, cetyl trimethylammonium bromide, polyvinyl pyrrolidone, and proteinase. In some embodiments, the DNA extracted from the lysed sample is bound to an affinity reagent (e.g. silica) in a binding buffer comprising proportional amounts of Tris-HCl, EDTA, and guanidine thiocyanate. In some embodiments, the DNA is serially washed in one or more buffers comprising Tris-HCl, EDTA, and ethanol, and eluted from the affinity reagent using an appropriate elution buffer.

Several methods exist for the isolation of DNA from bacterial cells. These methods essentially utilize the same basic procedure. In an exemplary embodiment, bacterial cells in a fecal sample are lysed enzymatically (i.e., lysozyme treatment), mechanically (i.e., bead homogenization) or by repeated freeze-thaw cycles, or combinations of these, followed by dissolution of the cell membrane with alkali and detergents such as sodium dodecyl sulfate (SDS) (Maniatis et al., 1989; Tsai et al., Appl. Environ. Microbiol., 57:1070-1074, 1991; Bej et al., Appl. Environ, Microbiol., 57:1013-1017, 1991). The cell lysate is then treated with proteinases and hexadecyltrimethyl ammonium bromide (CTAB) to degrade proteins and precipitate carbohydrates, respectively. The most common proteinase used in this procedure is proteinase K.

After extraction and physical separation of the DNA from other cellular components (lipid, carbohydrates, proteins), the DNA is isolated, or purified, according to methods known in the art. In certain embodiments, the DNA is isolated by a silica-based method, wherein the DNA is bound to a silica substrate, such as a silica membrane of silica beads, washed, and then eluted in isolated or purified form. In alternative embodiments, the DNA is isolated by phenol/chloroform extraction.

In some embodiments, the disclosure provides methods of extracting DNA from large quantities of fecal matter to enable detection of bacterial species present in low copy number. For example, methods are provided for isolating DNA from between about 0.5 g to about 1.0 g of fecal matter, and detecting a level of H. pylori present in the sample in as low as about 2 to about 5 copy numbers. In other embodiments, DNA is isolated from between about 0.01 g to about 0.1 g, about 0.1 g to about 0.5 g, between about 1.0 g to about 2 g of fecal matter. In some embodiments, the disclosure provides methods for detecting a level of H. pylori present in the sample in as low as about 2 copies, or as high as about 10 copies, about 15 copies, about 20 copies, or greater than 20 copies. In some embodiments, the disclosure provides methods for extracting total DNA present in a fecal sample.

Embodiments of the disclosure provide methods and compositions of identifying a low copy number DNA sequence in a fecal sample comprising hybridizing a labeled oligonucleotide probe to non-pathogenic bacterial DNA sequences in the DNA preparation to form a hybridization complex. In some embodiments, hybridizing a labeled probe comprises first denaturing the DNA in the DNA preparation. Conditions promoting denaturation, including high temperature and/or low ionic strength and/or moderate-to-high concentration of organic solvent, are well-known in the art. Similarly, conditions promoting hybridization, reannealing or renaturation, such as high ionic strength and/or lower temperatures, and the variation of these conditions to adjust the stringency of hybridization, are well-known in the art (e.g., Green et al., Sambrook et al., supra).

In embodiments, the labeled oligonucleotide probe of the disclosure is complementary to, and thus hybridizes with, a DNA sequence from a non-pathogenic bacteria. In some embodiments, the labeled oligonucleotide probe is complementary to a DNA sequence from Bacteroides, Clostridium, or Faecalibacterium. In embodiments, the labeled oligonucleotide probe is complementary to a DNA sequence from Bacteroides fragilis, Bacteroides melaninogenicus, Bacteroides oralis.

In embodiments, the labeled oligonucleotide of the disclosure comprises an oligonucleotide sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, or SEQ ID NO: 8. (Table 2.)

TABLE 2
Oligonucleotide Sequences of Embodiments of the
Disclosure

| SEQ | |
|---|---|
| ID | |
| NO: | Sequences |
| 1 | TTCCTCACATCTTACGACGGCAGTCTCTCCAGAGTCCTCAGCAT |
| | GACCTGTTAGTAACTGAAGATAAGGGTTGCGCTCGTTATGGCA |
| | CTTAAGCCGACA (SEQ ID NO: 1) |
| 2 | GGCACTTAAGCCGACACCTCACGGCACGAGCTGACGACAACCA |
| | TGCAGCACCTTCACAGCGGTGATTGCTCACTGACATGTTTCCAC |
| | ATCATTCCACTGC (SEQ ID NO: 2) |
| 3 | CACTTTCGAGCATCAGTGTCAGTTGCAGTCCAGTGAGCTGCCTT |
| | CGCAATCGGAGTTCTTCGTGATATCTAAGCATTTCACCGCTACA |
| | CCACGAATTCCG (SEQ ID NO: 3) |
| 4 | TATCTAATCCTGTTTGATACCCACACTTTCGAGCATCAGTGTCA |
| | GTTGCAGTCCAGTGAGCTGCCTTCGCAATCGGAGTTCTTCGTGA |
| | TATCTAAGCATT (SEQ ID NO: 4) |
| 5 | GCTCCCTTTAAACCCAATAAATCCGGATAACGCTCGGATCCTCC |
| | GTATTACCGCGGCTGCTGGCACGGAGTTAGCCGATCCTTATTCA |
| | TATTATACATAC (SEQ ID NO: 5) |
| 6 | TTGACGGGCGGTGTGTACAAGGCCCGGGAACGTATTCACCGCG |
| | CCGTGGCTGATGCGCGATTACTAGCGAATCCAGCTTCACGAAG |
| | TCGGGTTGCAGACT (SEQ ID NO: 6) |
| 7 | CCTCAGGTCATCCGGAAGCTTTTCAACGCTTATCGGTTCGGTCC |
| | TCCAGTTAGTGTTACCTAACCTTCAACCTGCCCAAGGGTAGATC |
| | ACTTGGTTTCGCGTCTACTCCTTCCGA (SEQ ID NO: 7) |
| 8 | TCACAGTACTGGTTCGCTATCGGTCTCTCGGGAGTATTTAGCCT |
| | TACCGGATGGTCCCGGCTGGTTCACGCAGAATTCCTCGTGCTCC |
| | GCGCTACTCAGGATACCACTAC (SEQ ID NO: 8) |

In embodiments, the oligonucleotide probe sequence of the disclosure comprises a sequence consisting of unmodified deoxynucleotides selected from deoxycytidine, deoxyadenosine, deoxyguanosine, and deoxythymidine. In some embodiments, the oligonucleotide probe sequence may be chemically modified. This may, for example, enhance their resistance to nucleases. For example, phosphorothioate oligonucleotides may be used. Other deoxynucleotide analogs include methylphosphonates, phosphoramidates, phosphorodithioates, N3 ‘P5’-phosphoramidates and oligoribonucleotide phosphorothioates and their 2′-O-alkyl analogs and 2′-O-methylribonucleotide methylphosphonates.

In embodiments, the oligonucleotide probe sequence of the disclosure comprises at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 21 nucleotides, at least 23 nucleotides, at least 24 nucleotides, at least 25 nucleotides, at least 26 nucleotides, at least 27 nucleotides, at least 28 nucleotides, at least 29 nucleotides, at least 30 nucleotides, at least 31 nucleotides, at least 32 nucleotides, at least 33 nucleotides, at least 34 nucleotides, at least 35 nucleotides, at least 36 nucleotides, at least 37 nucleotides, at least 38 nucleotides, at least 39 nucleotides, at least 40 nucleotides, at least 41 nucleotides, at least 42 nucleotides, at least 43 nucleotides, at least 44 nucleotides, at least 45 nucleotides, at least 46 nucleotides, at least 47 nucleotides, at least 48 nucleotides, at least 49 nucleotides, at least 50 nucleotides, at least about 55 nucleotides, at least about 60 nucleotides, at least about 65 nucleotides, at least about 70 nucleotides, at least about 75 nucleotides, or more.

It is also contemplated that the oligonucleotide probes of the disclosure may be used in combination. For example 1, 2, 3, 4, 5, 6, 7, 8, 9, 10, 11, 12, 13, 14, 15, 16, 17, 18, 19, 20, or more oligonucleotide probes are hybridized to non-pathogenic bacterial DNA sequences in the DNA preparation.

In embodiments, the labeled oligonucleotide probe of the disclosure is complementary to a conserved region of a non-pathogenic bacterial DNA sequence. As used herein, a conserved region comprises a sequence that exhibits homology or substantial sequence identity between and/or among bacterial species. Substantial sequence identity means a nucleic acid sequence has at least about 70 percent sequence identity as compared to a reference sequence, typically at least about 85 percent sequence identity, and preferably at least about 95 percent sequence identity as compared to a reference sequence. The percentage of sequence identity is calculated excluding small deletions or additions which total less than 25 percent of the reference sequence. The reference sequence may be a subset of a larger sequence, such as a portion of a gene or flanking sequence, or a repetitive portion of a chromosome. However, the reference sequence is at least 18 nucleotides long, typically at least about 30 nucleotides long, and preferably at least about 50 to 100 nucleotides long.

In some embodiments, the oligonucleotide probe sequence of the disclosure comprises a label. In exemplary embodiments, the label comprises an affinity tag that facilitates the physical separation of the oligonucleotide probe from other nucleic acids present in a sample. In embodiments, the label is added during synthesis, or after synthesis, of the oligonucleotide probe. In some embodiments, the label is directly coupled to, and thereby immobilized on, a solid support. In other embodiments, the label on the oligonucleotide probe is capable of being indirectly immobilized on a solid support, e.g., by an affinity reagent. In still other embodiments, the label is a peptide, a protein, an antibody, a glycoprotein, or a sugar.

In embodiments, the oligonucleotide probe sequence is labeled with an epitope recognized by an antibody or an antibody fragment. Accordingly, the epitope-labeled oligonucleotide, and hybridization complexes incorporating the epitope-labeled oligonucleotide, can be isolated by affinity purification methods, such as by immune-adsorption to a filter or column, or immunoprecipitation. Those skilled in the art will thus recognize that a label of the disclosure is capable of serving as, and is synonymous with, an affinity tag. In still further embodiments, the label is a peptide, a protein, an antibody, a glycoprotein, or a sugar. In certain embodiments, the oligonucleotide probe sequence is labeled with biotin.

In some embodiments, the oligonucleotide probe sequence is labeled with a plurality of labels. Thus, for example, embodiments provide an oligonucleotide probe that incorporates an affinity label in conjunction with one or more reporter groups, or detection reagents. In embodiments, a label comprises a fluorochrome (or fluorescent compounds), an enzyme (e.g., alkaline phosphatase or horseradish peroxidase), heavy metal chelates, secondary reporters or radioactive isotopes.

The labels of the disclosure can be linked to an oligonucleotide probe by methods known in the art. In some embodiments, a label is covalently added to either 5′ or 3′ terminal ribose positions. In embodiments, a label is added to a 5′ or 3′ terminal ribose modified with a chemical moiety suitable for covalently linking the oligonucleotide probe to a label. In other embodiments, the label comprises a modified nucleotide triphosphate that is incorporated during nucleotide synthesis. In still other embodiments, the label is added to an internucleotide linkage between bases of the oligonucleotide probe.

In embodiments, the disclosure further comprises depleting hybridization complexes formed between DNA sequences from a non-pathogenic bacterial species and a labeled oligonucleotide probe. In embodiments, depleting comprises denaturing the labeled oligonucleotide probe and the DNA preparation and allowing the probe sequence to hybridize (i.e. anneal) to the complementary bacterial DNA sequences. In some embodiments, the labeled oligonucleotide probe is immobilized to a solid substrate, such as a bead, column, or filter, prior to incubating the probe with the DNA preparation. Thus, in some embodiments the hybridization complexes form on the solid substrate. In other embodiments, the hybridization complexes are formed in solution and then incubated with a solute support bearing an affinity reagent that binds to the label on the oligonucleotide probe.

The affinity reagent on the solid support depends on the label on the oligonucleotide probe. In some embodiments, the solid support comprises an antigen and the label comprises an antibody, wherein the hybridization complexes bind to the antigen via the antibody. In other embodiments, the affinity reagent on the solid support is an antibody and the label is an epitope recognized by the antibody. In still further embodiments, the affinity reagent on the solid support is streptavidin, and the label on the probe is biotin.

In embodiments, depleting the labeled hybridization complexes comprises passing a solution over an affinity reagent immobilized on a solid support, wherein the hybridization complexes in solution are retained on the solid support via binding between the label and the affinity reagent and the remaining DNA preparation in the solution is collected. In other embodiments, depleting the hybridization complexes comprises binding to a bead coated with an affinity reagent, removing the beads by centrifugation, and collecting the supernatant solution. In still other embodiments, depleting the hybridization complexes comprises binding to a magnetic bead coated with an affinity reagent and removing the beads using a magnetic field.

In some embodiments, the labeled oligonucleotide of the disclosure comprises a non-affinity label. For example, in embodiments the oligonucleotide comprises a density label, a magnetic label, or a fluorometric label.

In other embodiments, the oligonucleotide label comprises a chemical moiety that reacts with a solid support structure and is thereby physically linked to the solid support. In some embodiments the chemical moiety is reversibly linked to the solid support. In exemplary embodiments, the oligonucleotide label comprises an amine group, a thiol group, an acrylic group, or alternative chemical moieties known in the art that are suitable for linking oligonucleotides to a solid support. X

The methods of the disclosure further comprise identifying low copy number DNA sequences in the DNA preparation depleted of non-pathogenic bacterial DNA sequence. In some embodiments, identifying a low copy number DNA sequence comprises a PCR reaction, such as quantitative PCR reaction. In some embodiments, the identifying a low copy DNA sequence comprises a sequencing reaction. In some embodiments, a library of low copy number DNA sequences are prepared and sequenced using next generation sequencing platforms, such as Illumina MiSeq or Thermo Fisher S5.

In some embodiments, the disclosure further provides methods for treating bacterial infection, such as H. pylori infection, in a subject. The methods may comprise: obtaining a sample from the subject, extracting DNA from the sample to obtain a DNA preparation, hybridizing a labeled oligonucleotide probe to non-pathogenic (i.e., non H. pylori) bacterial DNA sequences in the DNA preparation to form a hybridization complex, depleting the hybridization complex from the DNA preparation, identifying the presence of low copy number (i.e., H. pylori) DNA sequence in the DNA preparation, and administering to the subject one or more antibiotics.

In some embodiments, the disclosure provides methods for treating bacterial infection, such as H. pylori infection, further comprising amplifying a region of the H. pylori DNA to generate multiple copies of the amplified region of the H. pylori DNA, sequencing the multiple copies of the region of the H. pylori DNA, comparing sequences of the multiple copies of the region of the H. pylori DNA to one or more reference sequences, detecting a mutation in the multiple copies of the region of H. pylori DNA, determining a number of the multiple copies of the region of the H. pylori DNA with the mutation, wherein antibiotic resistant H. pylori is present in the sample when the number of the multiple copies of the region of the H. pylori DNA with the mutation is above a predetermined amount (e.g., the region of the H. pylori DNA with the mutation is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 98% or greater of the sequenced multiple copies of the region of the H. pylori DNA), and administering to the subject one or more antibiotics to which the H. pylori lacks resistance when antibiotic resistant H. Pylori is present in the sample.

The antibiotic resistant H. pylori may be resistant to one or more of the following: macrolides, metronidazole, quinolones, rifamycins, amoxicillin, and tetracycline.

As used herein, the terms, “treating” or “treatment” of a disease, disorder, or condition includes at least partially: (1) preventing the disease, disorder, or condition, i.e. causing the clinical symptoms of the disease, disorder, or condition not to develop in a mammal that is exposed to or predisposed to the disease, disorder, or condition but does not yet experience or display symptoms of the disease, disorder, or condition; (2) inhibiting the disease, disorder, or condition, i.e., arresting or reducing the development of the disease, disorder, or condition or its clinical symptoms; or (3) relieving the disease, disorder, or condition, i.e., causing regression of the disease, disorder, or condition or its clinical symptoms.

The present disclosure is illustrated in the following Examples, which are set forth to aid in understanding the invention, but should not be construed to limit in any way the scope of the disclosure as defined in the claims that follow.

### EXAMPLES

### Example 1: NGS Analysis of Low Copy Number H. pylori Antibiotic Resistance Genes

To determine the feasibility of detecting low copy number DNA sequences according to embodiments of the methods disclosed herein, an NGS-based sequence detection assay was performed. Genomic DNA of H. pylori strain 26695 was purchased from ATCC. To test the sensitivity of next generation sequencing in detecting the mutations of six genes associated with H. pylori antibiotic resistance, the 26695 genomic DNA was diluted to a series of copies from 1 million copies to 0.1 copy which then were used for library preparation. Each copy number dilution was performed in triplicate, with the exception of the 1 million copy and 100K copy samples, which were run in duplicate. The resulting libraries were sequenced using the Illumina® MiSeq® platform.

The sequencing data was analyzed with NGS analysis software NextGene® (SoftGenetics, State College, Pa.) by alignment with H. pylori 26695 reference sequence. The results show that from the 1 million copy sample down to the 10 copy sample produced 100% accurate sequence alignments (Table 3). Sequence errors were observed in the 5 copy to 0.1 copy samples (Table 4). For example, for the sequencing of 5 copies genomic DNA, there are sequencing errors in gyrA gene, AAT (wt) to ACC, GCG (wt) to GCA. The sensitivity of next generation sequencing in H. pylori antibiotic resistance analysis should be 10 copies or more of H. pylori DNA.

TABLE 3
NGS analysis of low copy number H. pylori antibiotic resistance genes:
template copy number = 1 × 10⁶ to 10
Copy numbers

| | 1M | | 100K | | 10K | | | 1000 | | | 100 | | | 10 | | |
|---|---|---|---|---|---|---|---|---|---|---|---|---|---|---|---|---|
| Pool | Rep1 | Rep2 | Rep1 | Rep2 | Rep1 | Rep2 | Rep3 | Rep1 | Rep2 | Rep3 | Rep1 | Rep2 | Rep3 | Rep1 | Rep2 | Rep3 |
| 16s rRNA-Tetracycline | S | NA | S | NA | S | S | S | S | S | S | S | S | S | S | S | S |
| 23s rRNA-Clarithromycin | S | NA | S | NA | S | S | S | S | S | S | S | S | S | S | S | S |
| gyrA-Fluoroquinolones | S | NA | S | NA | S | S | S | S | S | S | S | S | S | S | S | S |
| pbp-1a-Amoxycillin | S | NA | S | NA | S | S | S | S | S | S | S | S | S | S | S | S |
| rpoB-Rifampins | S | NA | S | NA | S | S | S | S | S | S | S | S | S | S | S | S |
| rdxA_Metronidazole | S | NA | S | NA | S | S | S | S | S | S | S | S | S | S | S | S |

S: Success,
F: Fail

TABLE 4
NGS analysis of low copy number H. pylori antibiotic resistance genes: template copy number = 5 to 0.1

| | 5 | | | 1 | | |
|---|---|---|---|---|---|---|
| Pool | Rep1 | Rep2 | Rep3 | Rep1 | Rep2 | Rep3 |
| 16s rRNA-Tetracycline | S | S | less than | S | S | S |
| | | | 250 pile-up | | | |
| 23s rRNA-Clarithromycin | S | S | S | S | S | less than |
| | | | | | | 250 pile-up |
| gyrA-Fluoroquinolones | [symbol] : AAT > ACC, | S | [symbol] : AAT > AAC | [symbol] : AAT > ACC, | S | [symbol] : AAT > ACC, |
| | GCG > GCA | | | GCG > GCA | | GCG > GCA |
| pbp-1a-Amoxycillin | S | S | S | S | S | S |
| rpoB-Rifampins | S | S | S | S | S | less than |
| | | | | | | 250 pile-up |
| rdxA_Metronidazole | S | S | S | S | S | S |
| | NGS Base Call (%) | | NGS Base Call (%) | NGS Base Call (%) | | NGS Base Call (%) |
| | AAT > ACC | | AAT > AAC | AAT > ACC | | AAT > ACC |
| | [symbol] | | [symbol] | [symbol] | | [symbol] |
| | GCG [symbol] GCA | | | GCG > GCA | | GCG > GCA |
| | [symbol] | | | [symbol] | | [symbol] |

| | 0.5 | | | 0.1 | | |
|---|---|---|---|---|---|---|
| Pool | Rep1 | Rep2 | Rep3 | Rep1 | Rep2 | Rep3 |
| 16s rRNA-Tetracycline | NA | S | less than | NA | S | less than |
| | | | 250 pile-up | | | 250 pile-up |
| 23s rRNA-Clarithromycin | NA | S | S | NA | S | less than |
| | | | | | | 250 pile-up |
| gyrA-Fluoroquinolones | NA | [symbol] : AAT > ACC, | [symbol] : AAT > AAC, | NA | [symbol] : AAT > ACC, | [symbol] : AAT > ACC |
| | | GCG > GCA | GCG > GCA | | GCG > GCA | |
| pbp-1a-Amoxycillin | NA | S | S | NA | S | S |
| rpoB-Rifampins | NA | S | [symbol] GTT > GCT | NA | S | S |
| rdxA_Metronidazole | NA | S | Not covered | NA | 5 | Not covered |
| | | NGS Base Call (%) | NGS Base Call (%) | | NGS Base Call (%) | NGS Base Call (%) |
| | | AAT > ACC | AAT > ACC | | AAT > ACC | AAT > ACC |
| | | [symbol] | [symbol] | | [symbol] | [symbol] |
| | | GCG > GCA | GCG > GCA | | GCG > GCA | |
| | | [symbol] | [symbol] | | [symbol] | |
| | | | GTT > GCT | | | |
| | | | [symbol] | | | |

[symbol]

indicates data missing or illegible when filed

### Example 2: NGS Analysis of Fecal Genomic DNA and Bacterial Controls

Total genomic DNA was extracted from fecal samples infected with Salmonella (detected by Luminex), and controls comprising buffer inoculated with control bacteria (Campylobacter, Salmonella, Shigella, Vibrio, Yersinia enterolitica and Shiga-toxin producing E. coli). To detect bacteria, viruses, fungi, protists, etc. in the microbial community—including antibiotic resistance strains—shotgun metagenomics was performed targeting all DNA material in the sample to assess relative abundance information for all sequences analyzed. Shotgun metagenomics provides a base-pair level resolution of the genome and makes single nucleotide variant analysis possible for antibiotic resistance analysis and for strain identification.

Shotgun metagenomics library preparation includes DNA fragmentation, end repair and A-tailing, adapter ligation and library amplification combining enzymatic steps and bead-based cleanups. The resulting libraries are sequenced on the Illumina MiSeq NGS platform.

Shotgun metagenomics data analysis was performed with assembly which involves the merging of reads from the same genome into a single contiguous sequence. After sequence assembly, genes are predicted and functionally annotated.

Shotgun metagenomics of the fecal sample generated a total 7,624,876 reads, of which 1,627,952 reads hit to bacteria. Controls generated 949,199 reads, of which 31,030 reads hits to bacteria (Table 5). Further analysis detected 293 bacteria, 2 fungi, 4 protists, 97 viruses, 4 respiratory viruses and virulence factors as well as antibiotic resistance mutations in fecal samples. In the buffer control, all of the spiked bacteria were detected and the hits include 45 bacteria, 4 fungi, 4 protists 63 viruses, 161 virulence factors and 64 antibiotic resistance mutations.

TABLE 5
NGS analysis of fecal genomic DNA and bacterial controls

| | # of | # of bacterial | | | |
|---|---|---|---|---|---|
| Sample Type | reads | hits | Name | Hits | Concordance |
| Fecal sample | 7,624,876 | 1,627,952 | Bacteria | 293 | Samonella detected |
| with Salmonella infection | | | Antibiotic | 80 | |
| | | | Resistance | | |
| | | | Fungi | 2 | |
| | | | Protists | 4 | |
| | | | Viruses | 97 | |
| | | | Respiratory Virus | 4 | |
| | | | Virulence Factors | 125 | |
| Buffer with inoculated | 949,199 | 31,030 | Bacteria | 45 | Campylobacter (0.3%) |
| bacteria | | | Antibiotic | 64 | Samonella (0.93%) |
| Campylobacter, Salmonella, | | | Resistance | | |
| Shigella, Vibrio, Yersinia | | | Fungi | 4 | Shigella (19.5%) |
| enterolitica, Shiga Toxin 1/2 | | | Protists | 4 | Vibrio (11.1%) |
| | | | Viruses | 63 | Yersinia (2.8%) |
| | | | Respiratory Virus | 0 | E coli (58%)- Shiga |
| | | | | | toxins |
| | | | Virulence Factors | 161 | |

It is to be understood that the embodiments of the disclosure are illustrative of the principles of the present disclosure. Other modifications that can be employed are within the scope of the disclosure. Thus, by way of example, but not of limitation, alternative configurations of the present disclosure can be utilized in accordance with the teachings herein. Accordingly, the present disclosure is not limited to that precisely as shown and described.

While the present disclosure has been described and illustrated herein by references to various specific materials, procedures and examples, it is understood that the disclosure is not restricted to the particular combinations of materials and procedures selected for that purpose. Numerous variations of such details can be implied as will be appreciated by those skilled in the art. It is intended that the specification and examples be considered as exemplary only, with the true scope and spirit of the disclosure being indicated by the following claims. All references, patents, and patent applications referred to in this application are herein incorporated by reference in their entirety.

1. A method of identifying a low copy number DNA sequence in a fecal sample comprising:
obtaining the fecal sample from a subject,
extracting DNA from the fecal sample to obtain a DNA preparation,
hybridizing a labeled oligonucleotide probe to non-pathogenic bacterial DNA sequences in the DNA preparation to form a hybridization complex,
depleting the hybridization complex from the DNA preparation, and
identifying the presence of the low copy number DNA sequence in the DNA preparation,
wherein identification of the low copy number DNA sequence in the DNA preparation indicates that the low copy number DNA sequence is present in the fecal sample.

2. The method of claim 1, wherein the low copy number DNA sequence is a H. pylori DNA sequence.

3. The method of claim 1, wherein the low copy number DNA sequence is a human DNA sequence.

4. The method of claim 3, wherein the human DNA sequence is associated with a cancerous or precancerous condition.

5. The method of claim 1, wherein the non-pathogenic bacterial DNA is from Bacteroides, Clostridium, Faecalibacterium, or a combination thereof.

6. The method of claim 1, wherein the labeled oligonucleotide probe is complementary to a conserved region of the non-pathogenic bacterial DNA.

7. The method of claim 1, wherein the label is biotin.

8. The method of claim 7, further comprising incubating the biotin-labeled hybridization complex with a streptavidin-coated substrate.

9. The method of claim 8, wherein the streptavidin-coated substrate comprises a bead, a column, or a membrane.

10. The method of claim 8, wherein the streptavidin-coated substrate comprises a magnetic bead, and wherein the hybridization complexes are depleted from the DNA preparation using a magnetic field.

11. The method of claim 1, wherein the hybridization complexes are depleted from the DNA preparation using centrifugal force.

12. The method of claim 1, wherein the non-pathogenic bacterial DNA is from Bacteroides fragilis, Bacteroides melaninogenicus, Bacteroides oxalis, or a combination thereof.

13. The method of claim 12, wherein the labeled oligonucleotide probe is selected from SEQ ID NO: 1, SEQ ID NO: 2, SEQ ID NO: 3, SEQ ID NO: 4, SEQ ID NO: 5, SEQ ID NO: 6, SEQ ID NO: 7, or SEQ ID NO: 8.

14. The method of claim 1, wherein identifying a low copy number DNA sequence further comprises sequencing the DNA sequences.

15. The method of claim 1, wherein identifying a low copy number DNA sequence comprises a quantitative PCR reaction.

16. The method of claim 1, wherein identifying a low copy number DNA sequence comprises multiplexing the sample with one or more additional samples.

17. The method of claim 1, wherein DNA extracted from the fecal sample weighs between about 0.5 grams to about 1.0 grams.

18. The method of claim 1, wherein total DNA is extracted from the fecal sample.

19. The method of claim 1, wherein extracting the DNA comprises bead homogenizing the fecal sample in a lysis buffer, wherein the lysis buffer comprises ingredients capable of breaking a bacterial cell wall, digesting protein, denaturing protein, dispersing fat, precipitating polysaccharides, or a combination thereof.

20. A method of enriching low copy number DNA sequences in a fecal sample comprising:
obtaining the fecal sample from a subject,
extracting DNA from the fecal sample to obtain a DNA preparation,
hybridizing a labeled oligonucleotide probe to non-pathogenic bacterial DNA sequences in the DNA preparation to form a hybridization complex,
depleting the hybridization complex from the DNA preparation.

21. A method of identifying antibiotic resistant H. pylori in a fecal sample comprising:
obtaining the fecal sample from a subject,
extracting DNA from the fecal sample to obtain a DNA preparation,
hybridizing a labeled oligonucleotide probe to non-pathogenic bacterial DNA sequences in the DNA preparation to form a hybridization complex,
depleting the hybridization complex from the DNA preparation,
amplifying a region of H. pylori DNA in the DNA preparation to generate multiple copies of the region of the H. pylori DNA,
sequencing the multiple copies of the amplified region of the H. pylori DNA,
comparing sequences of the multiple copies of the amplified region of the H. pylori DNA to a reference sequence,
identifying the presence of a mutation in the multiple copies of the region of the H. pylori DNA, and
determining a number of the multiple copies of the region of the H. pylori DNA with the mutation,
wherein antibiotic resistant H. pylori is present in the sample when the number of the multiple copies of the region of the H. pylori DNA with the mutation is above a predetermined amount.

22. (canceled)

23. (canceled)
